(19) United States
(12) Patent Application Publication
(10) Pub. No.: US 2020/0178844 A1
(43) Pub. Date: Jun. 11, 2020

(54) CONDITION CHANGE DETERMINATION METHOD, CONDITION CHANGE DETERMINATION APPARATUS, AND PROGRAM AND COMPUTER READABLE MEDIUM

(71) Applicant: NIHON KOHDEN CORPORATION, Tokyo (JP)

(72) Inventors: Takayuki SUGIYAMA, Tokorozawa-shi (JP); Akihiko YOSHIMURA, Tokorozawa-shi (JP); Kazuri HIGASHI, Tokorozawa-shi (JP)

(73) Assignee: NIHON KOHDEN CORPORATION, Tokyo (JP)

(21) Appl. No.: 16/704,114

(22) Filed: Dec. 5, 2019

(30) Foreign Application Priority Data
Dec. 6, 2018 (JP) 2018-229053

(51) Int. Cl. A61B 5/08; A61B 5/085; A61B 5/00

(52) U.S. Cl. CPC A61B 5/0826; A61B 5/0816; A61B 5/742; A61B 5/746; A61B 5/085

(57) ABSTRACT

A condition change determination method and condition change determination apparatus that, when a medical person uses a method for determining a condition change of a patient, such as NEWS, can accurately determine the condition of the patient, and a program and computer readable medium that are to be used in the apparatus or the method are provided. A non-transitory computer readable medium including a program causes a computer to realize the functions of: acquiring at least one of apnea and hypopnea rates of a patient; acquiring vital signs information of the patient other than the apnea and hypopnea rates; and determining a condition change of the patient by a determination method for determining a condition change of a patient based on at least one of the apnea and hypopnea rates, and the vital signs information other than the apnea and hypopnea rates.

### CROSS-REFERENCE TO RELATED APPLICATION

This application claims priority from Japanese Patent Application No. 2018-229053, filed on Dec. 6, 2018, the entire subject matter of which is incorporated herein by reference.

### TECHNICAL FIELD

The presently disclosed subject matter relates to a condition change determination method, a condition change determination apparatus, and a program and computer readable medium that are to be used in the apparatus or the method.

### BACKGROUND

As a method for determining a condition change of a patient, conventionally, techniques such as NEWS (National Early Warning Score) and qSOFA (quick SOFA) are known. The respiration rate is used as one of evaluation items (parameters) that are used in NEWS and qSOFA (see JP-T-2015-522822).

### SUMMARY

For example, the impedance method using an electrocardiogram is known as a method for measuring the respiration rate. However, the method is susceptible to noise such as artifact, and therefore it is difficult to accurately measure the respiration rate. When the respiration rate of a patient is to be measured, therefore, a medical person visually measures the respiration rate of the patient.

It is a primary object of the presently disclosed subject matter to provide a condition change determination method and condition change determination apparatus that, when a medical person uses a method for determining a condition change of a patient, such as NEWS, can accurately determine the condition of the patient, and also to provide a program and computer readable medium that are to be used in the apparatus or the method.

A non-transitory computer readable medium including a program of a mode of the presently disclosed subject matter causes a computer to realize the functions of:

acquiring at least one of apnea and hypopnea rates of a patient;

acquiring vital signs information of the patient other than the apnea and hypopnea rates; and

determining a condition change of the patient by a determination method for determining a condition change of a patient based on at least one of the apnea and hypopnea rates, and the vital signs information other than the apnea and hypopnea rates.

### BRIEF DESCRIPTION OF DRAWINGS

FIG. 1 is a functional block diagram of a condition change determination apparatus of an embodiment of the presently disclosed subject matter, and a bedside monitor.

FIG. 2 illustrates reference values of a determination method based on NEWS that is one of determination methods.

FIG. 3 illustrates reference values of a determination method based on SOFA that is one of determination methods.

FIG. 4 illustrates reference values of a determination method based on qSOFA that is one of determination methods.

FIG. 5 illustrates a normalization table for the determination methods.

FIG. 6 illustrates a normalization table for normalizing parameters constituting the determination methods.

FIG. 7 illustrates a flowchart relating to a condition change determination method of an embodiment of the presently disclosed subject matter.

FIG. 8 illustrates a flowchart relating to setting of a first time interval.

FIG. 9 is a view relating to a screen that displays condition changes of patients.

FIG. 10 is a functional block diagram of a condition change determination apparatus of an embodiment of the presently disclosed subject matter, and a display device.

### DESCRIPTION OF EMBODIMENTS

Hereinafter, embodiments of the presently disclosed subject matter will be described with reference to the accompanying drawings.

### First Embodiment

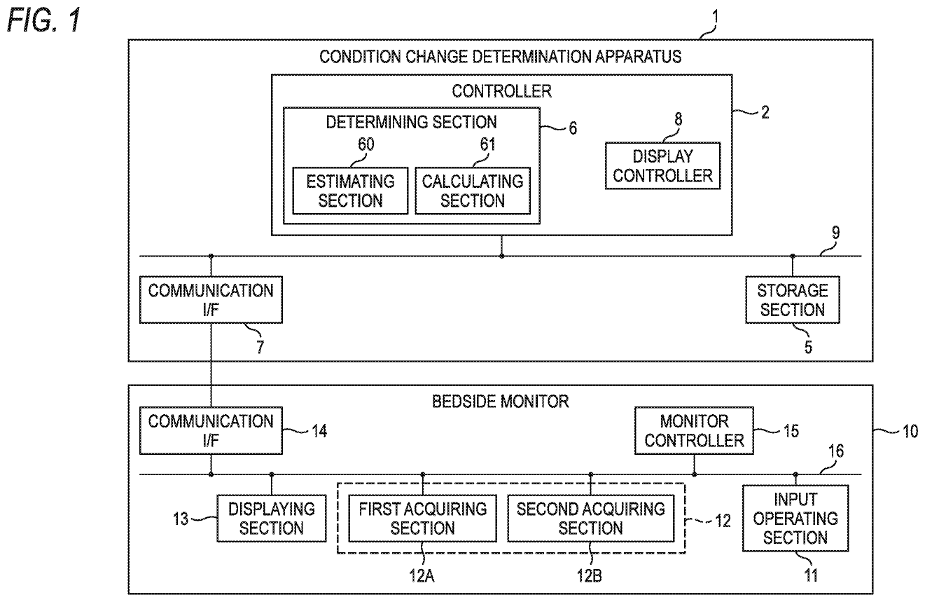

FIG. 1 is a functional block diagram of a condition change determination apparatus 1 of a first embodiment, and a bedside monitor (an example of an external apparatus) 10. As illustrated in FIG. 1, the condition change determination apparatus 1 may include a controller 2, a storage section 5, and a communication interface (communication I/F) 7. These components are communicably connected to one another via a bus 9. The bedside monitor 10 may include an input operating section 11, an acquiring section 12, a displaying section 13, a communication interface (communication I/F) 14, and a monitor controller 15. These components are communicably connected to one another via a bus 16. The condition change determination apparatus 1 and the bedside monitor 10 are communicable with each other in a wired or wireless manner.

The condition change determination apparatus 1 will be described. The controller 2 may include a determining section 6 and a display controller 8. The hardware configuration of the controller 2 may further include a memory and a processor. The memory is configured by, for example, a ROM (Read Only Memory) in which various programs and the like are stored, and a RAM (Random Access Memory) having a plurality of work areas in which various programs to be executed by the processor, and the like are stored. For example, the processor is a CPU (Central Processing Unit), and configured so as to load designated ones of the various programs incorporated in the ROM, into the RAM, and execute various processes in cooperation with the RAM. When the controller 2 executes programs in cooperation with the RAM, for example, the controller controls processes of the determining section 6 or the display controller 8 to be realized. Furthermore, the controller 2 controls the determining section 6 so as to perform a predetermined process based on vital signs information that is acquired by the acquiring section 12 of the bedside monitor 10.

In the embodiment, a computer readable medium may be used. A computer readable medium means all kinds of physical memories (a RAM, a ROM, and the like) that can store information and data which can be read by a processor. The computer readable medium can store instructions relating to processes to be executed by one or more processors. It should be understood that the term “computer readable medium” includes tangible items, and excludes a carrier wave and a transitory signal (namely, indicates a non-transitory medium).

The storage section 5 may include a memory for storing determination methods for determining a condition change of a patient, reference values and allowable ranges that are defined in medical guidelines or the like relating to the condition change determination method, a normalization table (an example of normalization conditions) for normalizing determination results, and the like. Examples of the condition change determination methods are NEWS, SOFA, qSOFA, APACHE2 (Acute Physiology and Chronic Health Evaluation 2), BSAS (Bedside Shivering Assessment Scale), and NIHSS (National Institutes of Health Stroke Scale). The condition change determination methods that are stored in the storage section 5 may include, for example, a determination method that is set based on NEWS or the like, in addition to the above-mentioned determination methods.

The communication interface 7 is an interface that enables the apparatus to communicate with the bedside monitor 10. The condition change determination apparatus 1 can appropriately communicate with the bedside monitor 10 via the communication interface 7.

The display controller 8 produces image data for displaying the result of the determination performed by the determining section 6, on the displaying section 13 of the bedside monitor 10 in a display mode that is normalized based on a normalization table P or normalization table Q stored in the storage section 5. In the normalization method in the embodiment, a color is applied to a predetermined place. The image data transmitted to the bedside monitor 10. As described above, the display controller 8 can control the displaying section 13 of the bedside monitor 10.

The bedside monitor 10 will be described. The input operating section 11 is configured so as to receive an input operation performed by the medical person who operates the bedside monitor 10, and produce an instruction signal corresponding to the input operation. For example, the input operating section 11 is configured by a touch panel that is overlaid on the displaying section 13, or operation buttons or event switches that are mounted on a case. The input operating section 11 receives an input operation for starting acquisition of vital signs information, that is for switching screens of the displaying section 13, and the like, and produces an instruction signal corresponding to the input operation. The produced instruction signal is transmitted to the monitor controller 15 via the bus 16. The monitor controller 15 is configured so as to control the operation of the bedside monitor 10 based on the instruction signal. The produced instruction signal is also transmitted to the controller 2 of the condition change determination apparatus 1 via the bus 16 and the communication interface 14, and the communication interface 7 and the bus 9. The controller 2 is configured so as to control the operation of the condition change determination apparatus 1 based on the instruction signal. Alternatively, the input operating section 11 may be disposed in the condition change determination apparatus 1.

The acquiring section 12 is configured so as to be able to acquire vital signs information of the patient. For example, the acquiring section 12 is an interface to which a plurality of electrodes, cuff, and the like that are to be attached to the patient are to be connected. Examples of vital signs information are the respiration rate, the blood pressure, the body temperature, the heart rate, and the like. The vital signs information of the patient that is acquired by the acquiring section 12 is transmitted to the storage section 5 of the condition change determination apparatus 1. In the specification, quantifiable vital signs information may be sometimes referred to as a measurement value. The acquiring section 12 may be disposed in the condition change determination apparatus 1.

The bedside monitor 10 may further acquire vital signs information via the input operating section 11. When the medical person confirms that the patient is unconscious, for example, the medical person inputs information indicating that the patient is unconscious, in the input operating section 11. The input operating section 11 transmits the input information to the monitor controller 15.

The acquiring section 12 may include a first acquiring section 12A and a second acquiring section 12B. The first acquiring section 12A is configured so as to acquire the apnea and hypopnea rates of the patient. The second acquiring section 12B is configured so as to acquire vital signs information other than the apnea and hypopnea rates of the patient. Examples of the vital signs information other than the apnea and hypopnea rates are the respiration rate, the blood pressure, the body temperature, the heart rate, and the like. In the specification, the term “acquiring section” has the meaning including the first acquiring section and the second acquiring section.

Examples of the method of measuring the respiration rate are a method in which the respiration rate is visually measured by a medical person, the impedance method using an electrocardiogram, and a measurement method using the carbon dioxide concentration (EtCO2). The measurement time period in the case where the respiration rate is measured by the impedance method using an electrocardiogram, or the measurement method using the carbon dioxide concentration (EtCO2) is shorter than that in the case where the respiration rate is visually measured by a medical person.

The displaying section 13 is a display device such as a liquid crystal display or an organic EL display, and configured so as to display the result of the determination performed by the determining section 6. Alternatively, the displaying section 13 may be disposed in the condition change determination apparatus 1, or in another display device other than the condition change determination apparatus 1 and the bedside monitor 10. Examples of the display device are a display unit and a mobile terminal.

The monitor controller 15 may be configured in the same or similar manner as the controller 2. For example, the monitor controller 15 executes programs in cooperation with a RAM, thereby realizing processes of the acquiring section 12.

The communication interface 14 is an interface that enables the bedside monitor to communicate with the condition change determination apparatus 1. The bedside monitor 10 can appropriately communicate with the condition change determination apparatus 1 via the communication interface 14.

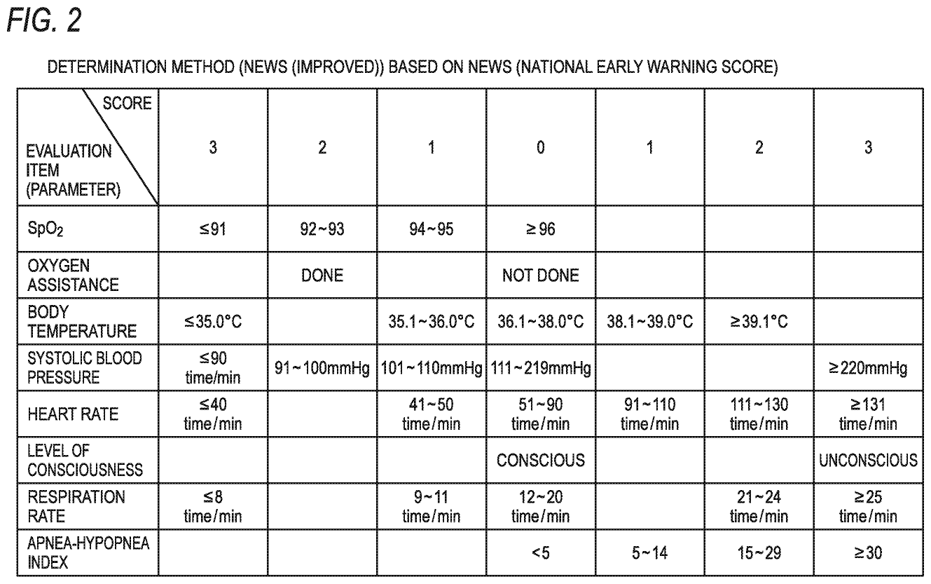

FIG. 2 illustrates reference values of a determination method (NEWS (improved)) based on NEWS that is one of the determination methods. The determination method is set based on NEWS that is used in determination of septicemia. The determination method is different from conventional NEWS in that the apnea-hypopnea index is added in addition to the evaluation items (parameters) that are used in NEWS. Namely, the evaluation items (parameters) that are used in the determination method are the transcutaneous arterial oxygen saturation (SpO₂), oxygen assistance, the body temperature, the systolic blood pressure, the heart rate, the level of consciousness, the respiration rate, and the apnea-hypopnea index. The apnea-hypopnea index is a value that is calculated by converting the total number of the apnea rate and hypopnea rate during sleep, to the mean rate per hour. In the case where septicemia is to be determined by using the determination method, when the total number of the apnea and hypopnea rates per hour that are acquired by the first acquiring section 12A is less than 5, for example, the score relating to the apnea-hypopnea index is 0. When the total number of the apnea and hypopnea rates is 5 or more and 14 or less, the score relating to the apnea-hypopnea index is 1. When the total number of the apnea and hypopnea rates is 15 or more and 29 or less, the score relating to the apnea-hypopnea index is 2. When the total number of the apnea and hypopnea rates is 30 or more, the score relating to the apnea-hypopnea index is 3. When the respiration rate per minute that is acquired by the second acquiring section 12B is 8 or less or 25 or more, the score relating to the respiration rate is 3. When the respiration rate is 21 or more and 24 or less, the score relating to the respiration rate is 2. When the respiration rate is 9 or more and 11 or less, the score relating to the respiration rate is 1. When the respiration rate is 12 or more and 20 or less, the score relating to the respiration rate is 0. Also with respect to the other parameters, scores are calculated based on reference values or the like that are defined in respective medical guidelines or the like. The calculated scores are totalized. In the specification, the totalized score is referred to as the combined score A.

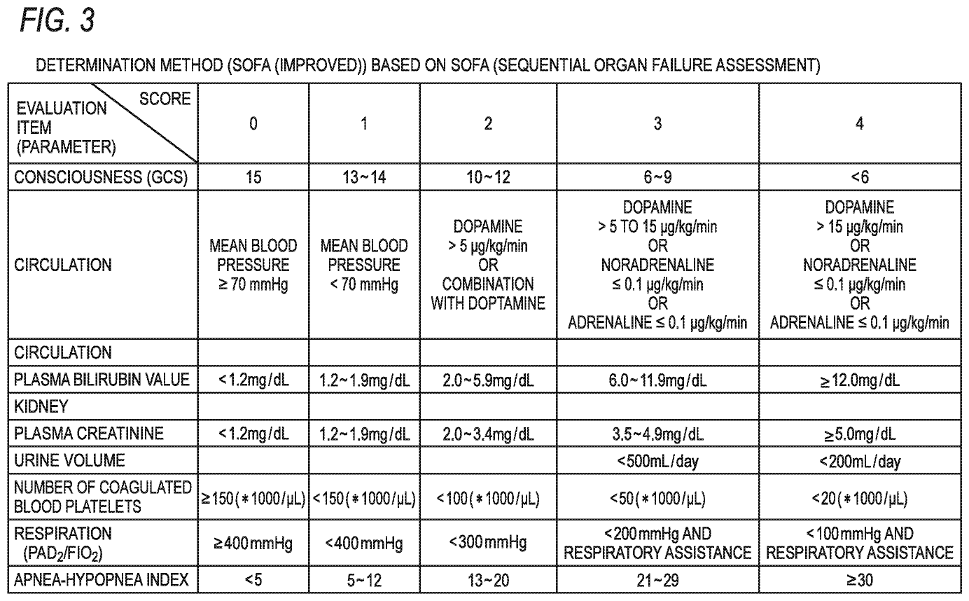

FIG. 3 illustrates reference values of a determination method (SOFA (improved)) based on SOFA that is one of the determination methods. SOFA is a method for determining septicemia. Particularly, SOFA is used for determining whether there is organ failure or not. The determination method is different from conventional SOFA in that the apnea-hypopnea index is added in addition to the evaluation items (parameters) that are used in SOFA. Namely, the evaluation items (parameters) that are used in the determination method are the consciousness, the circulation, the plasma bilirubin value, the plasma creatinine value, the number of coagulated blood platelets, the respiration, and the apnea-hypopnea index. In the case where septicemia is to be determined by using the determination method, when the total number of the apnea and hypopnea rates per hour that are acquired by the first acquiring section 12A is less than 5, for example, the score relating to the apnea-hypopnea index is 0. When the total number of the apnea and hypopnea rates is 5 or more and 12 or less, the score relating to the apnea-hypopnea index is 1. When the total number of the apnea and hypopnea rates is 13 or more and 20 or less, the score relating to the apnea-hypopnea index is 2. When the total number of the apnea and hypopnea rates is 21 or more and 29 or less, the score relating to the apnea-hypopnea index is 3. When the total number of the apnea and hypopnea rates is 30 or more, the score relating to the apnea-hypopnea index is 4. When the number of coagulated blood platelets that is acquired by the second acquiring section 12B is less than 20, the score relating to the number of coagulated blood platelets is 4. When the number of coagulated blood platelets is 20 or more and less than 50, the score relating to the number of coagulated blood platelets is 3. When the number of coagulated blood platelets is 50 or more and less than 100, the score relating to the number of coagulated blood platelets is 2. When the number of coagulated blood platelets is 100 or more and less than 150, the score relating to the number of coagulated blood platelets is 1. When the number of coagulated blood platelets is 150 or more, the score relating to the number of coagulated blood platelets is 0. Also with respect to the other items, scores are calculated based on reference values or the like that are defined in respective medical guidelines or the like. The calculated scores are totalized. In the specification, the totalized score is referred to as the combined score B.

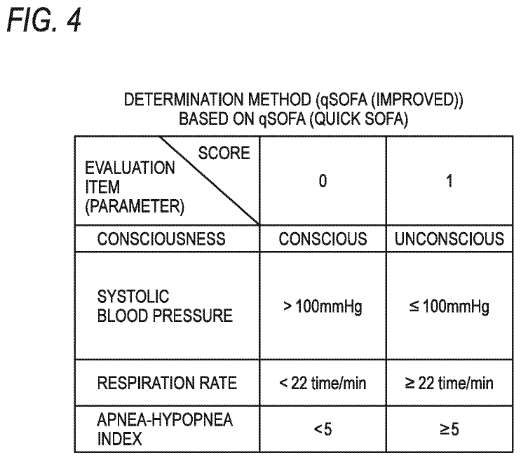

FIG. 4 illustrates reference values of a determination method (qSOFA (improved)) based on qSOFA that is one of the determination methods. qSOFA is a method for determining septicemia. Particularly, qSOFA is used for determining whether the patient is affected with an infectious disease or not. The determination method is different from conventional qSOFA in that the apnea-hypopnea index is added in addition to the evaluation items (parameters) that are used in qSOFA. Namely, the evaluation items (parameters) that are used in the determination method are the systolic blood pressure, the consciousness, the respiration rate, and the apnea-hypopnea index. When the total number of the apnea and hypopnea rates per hour that are acquired by the first acquiring section 12A is less than 5, for example, the score relating to the apnea-hypopnea index is 0. When the total number of the apnea and hypopnea rates is 5 or more, the score relating to the apnea-hypopnea index is 1. When the respiration rate per minute that is acquired by the second acquiring section 12B is 22 or more, the score relating to the respiration rate is 1. When none of the above applies, the score is 0. When the systolic blood pressure that is acquired by the second acquiring section 12B is 100 mmHg or less, the score relating to the systolic blood pressure is 1. When the above does not apply, the score is 0. When the patient is unconscious, the score relating to the consciousness is 1. When the patient is conscious, by contrast, the score relating to the consciousness is 0. The calculated scores are totalized. In the specification, the totalized score is referred to as the combined score C.

In the specification, the values, percentages, and the like that are calculated by the determining section 6 based on the vital signs information acquired by the acquiring section 12 and the determination methods stored in the storage section 5, such as the combined score A, the combined score B, and the combined score C are referred to as the combined score.

In methods for determining septicemia, as described above, the medical meaning of the reference values and the value of the combined score are different depending on the used determination method.

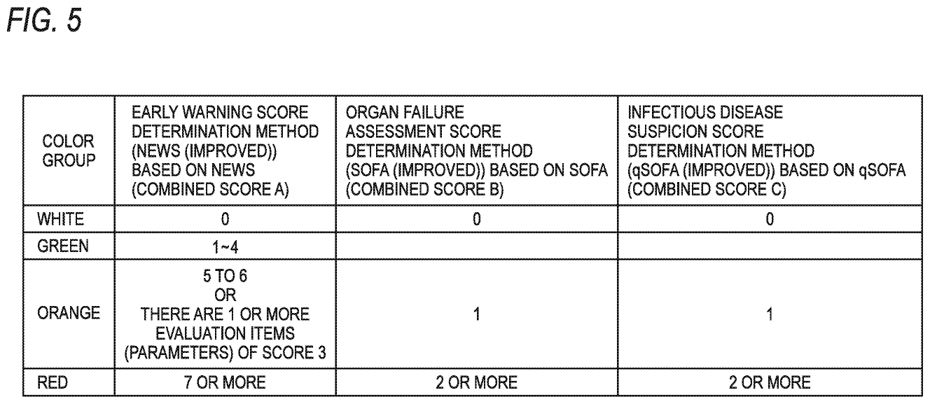

FIG. 5 illustrates a normalization table P in the determination methods. The normalization table P is a table for displaying condition level while classifying the condition levels of the patient into four color groups for each of the determination methods. In the embodiment, the four color groups are a white group, a green group, an orange group, and a red group. The white group corresponds to a condition level in which the patient is in the normal condition. The red group corresponds to a condition level in which the patient is in an abnormal condition, and in a critical condition. The orange group corresponds to a condition level in which the patient is not so critical as the patient of the red group, and is not in the normal condition. The green group corresponds to a condition level in which the patient is not so severe as the patient of the orange group, and is not in the normal condition. Namely, the patient condition levels other than the white group are severer in the sequence of the green group, the orange group, and the red group. The classification of the color groups that is described here is a mere example. It is a matter of course that the color group classification is not limited to the example.

With respect to the early warning score (NEWS (improved)), when the combined score A is 0, for example, the color group is classified into the white group. When the combined score A is 1 or more and 4 or less, the color group is classified into the green group. When the combined score A is 5 or more and 6 or less, or when the score of at least one of the evaluation items in NEWS is 3, the color group is classified into the orange group. When the combined score A is 7 or more, the color group is classified into the red group.

With respect to the organ failure assessment score (SOFA (improved)), when the combined score B is 0, the color group is classified into the white group. When the combined score B is 1, the color group is classified into the orange group. When the combined score B is 2 or more, the color group is classified into the red group.

With respect to the infectious disease suspicion score (qSOFA (improved)), when the combined score C is 0, the color group is classified into the white group. When the combined score C is 1, the color group is classified into the orange group. When the combined score C is 2 or more, the color group is classified into the red group.

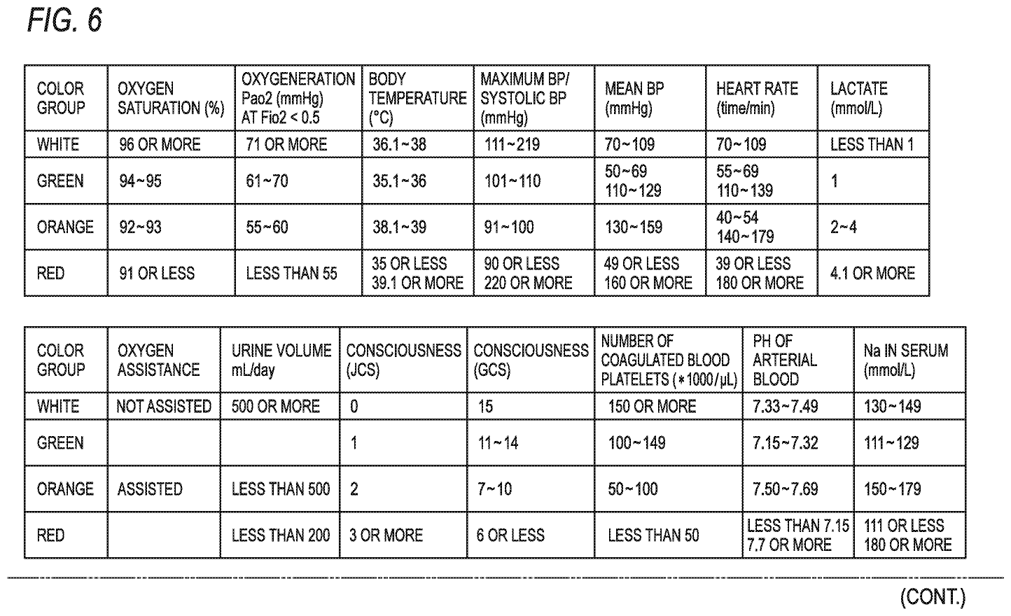

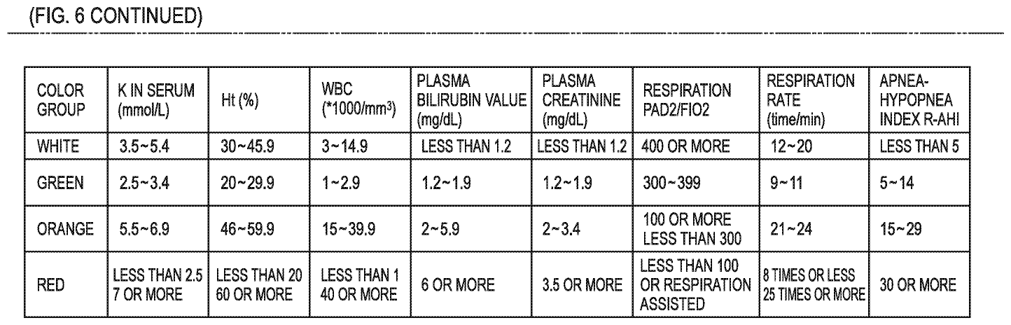

FIG. 6 illustrates a normalization table Q for normalizing parameters constituting the determination methods. As illustrated in FIG. 6, when the respiration rate per minute is 12 or more and 20 or less, for example, the color group is classified into the white group. When the respiration rate is 9 or more and 11 or less, the color group is classified into the green group. When the respiration rate is 21 or more and 24 or less, the color group is classified into the orange group. When the respiration rate is 8 or less or 25 or more, the color group is classified into the red group. With respect to the apnea-hypopnea index, when the apnea-hypopnea index is less than 5, the color group is classified into the white group. When the apnea-hypopnea index is 5 or more and 14 or less, the color group is classified into the green group. When the apnea-hypopnea index is 15 or more and 29 or less, the color group is classified into the orange group. When the apnea-hypopnea index is 30 or more, the color group is classified into the red group.

As illustrated in FIG. 6, with respect to oxygen assistance, in the case where oxygen assistance is required, the color group is classified into the orange group, and, in the case where oxygen assistance is not required, the color group is classified into the white group. With respect to the urine volume, when the urine volume per day is 500 mL or more, the color group is classified into the white group. When the urine volume per day is 200 mL or more and less than 500 mL, the color group is classified into the orange group. When the urine volume per day is less than 200 mL, the color group is classified into the red group. Each of the other parameters is classified into four stages, and the color groups are associated with the stages, respectively.

Returning to FIG. 1, the determining section 6 of the condition change determination apparatus 1 will be described. The determining section 6 may include an estimating section 60 and a calculating section 61. The estimating section 60 calculates an estimated value per time interval (second time interval) that is standard in the determination methods, based on the vital signs information which is acquired by the acquiring section 12 of the bedside monitor 10 at an time interval (first time interval) that is set by the medical person or the like. The second time interval is longer than the first time interval. Preferably, the first time interval is 1 minute or longer. In the specification, the first time interval in the case where the time interval is 1 minute is referred to as the minimum time interval. The calculating section 61 can calculate the scores of the parameters based on vital signs information that is acquired by the acquiring section 12 of the bedside monitor 10 at the first time interval, or the estimated value that is estimated by the estimating section 60. The calculating section 61 further calculates the combined score of the scores of each of the parameter. The determining section 6 is configured so as to determine a condition change of the patient based on the combined score calculated by the calculating section 61, and the determination methods stored in the storage section 5. In the case where the determination method based on NEWS, and that based on SOFA are used in order to determine whether a certain patient has septicemia or not, for example, the determining section 6 determines that the combined score A and combined score B that are calculated by the calculating section 61 correspond to which one of the above-described four color groups, based on the normalization table P stored in the storage section 5, and performs classification.

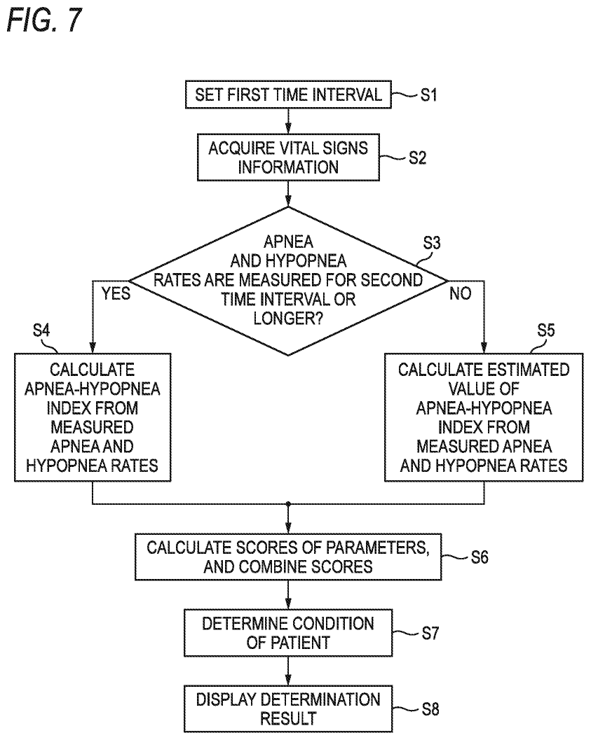

FIG. 7 illustrates a flowchart relating to a condition change determination method of the embodiment of the presently disclosed subject matter. The medical person or the like sets, through the input operating section 11, the time interval (first time interval) in which the first acquiring section 12A acquires the apnea and hypopnea rates of the patient (step S1). In the case where sleep apnea syndrome in the patient is to be determined by using vital signs information such as the apnea and hypopnea rates, vital signs information of the apnea and hypopnea rates is acquired over at least about one hour. Then, sleep apnea syndrome in the patient is determined based on the acquired vital signs information. Therefore, the determination of sleep apnea syndrome in the patient sometimes requires at least about one hour. When vital signs information of the apnea and hypopnea rates is to be used also in a method for determining a condition change of a patient other than the method for determining sleep apnea syndrome, at least about one hour is required to determine a condition change of the patient. However, in the case where determination whether a patient has septicemia or not is performed by using qSOFA, for example, it is determined whether the patient has septicemia or not, by using parameters of the patient, i.e., the consciousness, the systolic blood pressure, and the respiration rate. When the treatment of septicemia is early started, the patient is easily cured. Therefore, the parameters that are used in qSOFA are measured within several minutes. Therefore, there is a large difference between the measurement time period of the parameters that are used in a method for determining whether a patient has septicemia or not, such as qSOFA, and that of the parameters (the apnea and hypopnea rates) that are used in a method for determining whether a patient has sleep apnea syndrome or not. According to the embodiment, however, estimated values of the apnea and hypopnea rates can be calculated from the apnea and hypopnea rates that are measured during a measurement time period (the first time interval) which is shorter than the usual measurement time period for the apnea and hypopnea rates, and hence a condition change of a patient can be determined by using the apnea and hypopnea rates and another kind(s) of vital signs information. For example, the first time interval is set within a range of 1 to 60 minutes. Of course, the range of the first time interval is not limited to this range. The embodiment will be described under assumption that the first time interval is 15 minutes, and the second time interval is 60 minutes. When the first time interval is 15 minutes, the first acquiring section 12A acquires the apnea and hypopnea rates of the patient at an interval of 15 minutes. The second acquiring section 12B acquires vital signs information other than the apnea and hypopnea rate of the patient constantly or at a predetermined interval (step S2).

When these kinds of vital signs information are acquired, the controller 2 determines whether the apnea and hypopnea rates are measured for the second time interval (60 minutes) or longer or not (step S3). If YES in step S3, the calculating section 61 calculates the apnea-hypopnea index from the apnea and hypopnea rates that are acquired at the first time interval by the first acquiring section 12A (step S4). If NO in step S3, the estimating section 60 calculates an estimated value of the apnea-hypopnea index from the apnea and hypopnea rates that are acquired at the first time interval by the first acquiring section 12A (step S5). In the embodiment, the first time interval is 15 minutes. Therefore, step S5 is executed.

When the apnea rate is 0 within the first time interval, for example, the estimated value of the apnea index is 0. When the hypopnea rate is 1, the estimated value of the hypopnea index is 4. The apnea-hypopnea index is the total number of the apnea rate and the hypopnea rate. Therefore, the apnea-hypopnea index is 4. In the case where, during the first time interval, the apnea rate is 1, and the hypopnea rate is 1, the estimated value of the apnea index is 4, and that of the hypopnea index is 4. Therefore, the apnea-hypopnea index is 8. In the embodiment, as described above, the estimated value of the apnea-hypopnea index per the second time interval is calculated by using the apnea and hypopnea rates that are measured within the first time interval. Although the embodiment is described by way of the example in which the apnea-hypopnea index or an estimated value of the apnea-hypopnea index is calculated from the apnea and hypopnea rates, the invention is not limited to the example. For example, the apnea index or estimated value of the apnea index that is calculated from the apnea rate may be used in place of the apnea-hypopnea index. Furthermore, for example, the hypopnea index or estimated value of the hypopnea index that is calculated from the hypopnea rate may be used in place of the apnea-hypopnea index.

When the apnea-hypopnea index or an estimated value thereof is calculated, the calculating section 61 of the determining section 6 calculates the scores of the parameters and the combined scores (step S6). Alternatively, the calculating section 61 of the determining section 6 may calculate the scores of the parameters and the combined scores based on the apnea index or an estimated value of the apnea index, or the hypopnea index or an estimated value of the hypopnea index, in place of the apnea-hypopnea index. The determining section 6 determines a condition change of the patient based on the scores of the parameter and the normalization table Q, or the combined scores and the normalization table P (step S7). The scores of the parameters and combined scores that are calculated, and the determination result are stored in the storage section 5.

When the result of the determination performed by the determining section 6 is stored in the storage section 5, the display controller 8 produces image data for displaying the result of the determination performed by the determining section 6 in a display mode that is normalized based on the normalization table P or the normalization table Q, on the displaying section 13 of the bedside monitor 10. The image data are transmitted to the bedside monitor 10. As a result, an image based on the image data is displayed on the displaying section 13. Namely, the determination result is displayed on the displaying section 13 in the normalized display mode (step S8). As described above, the display controller 8 controls the displaying section 13 so as to display the result of the determination performed by the determining section 6, in the normalized display mode.

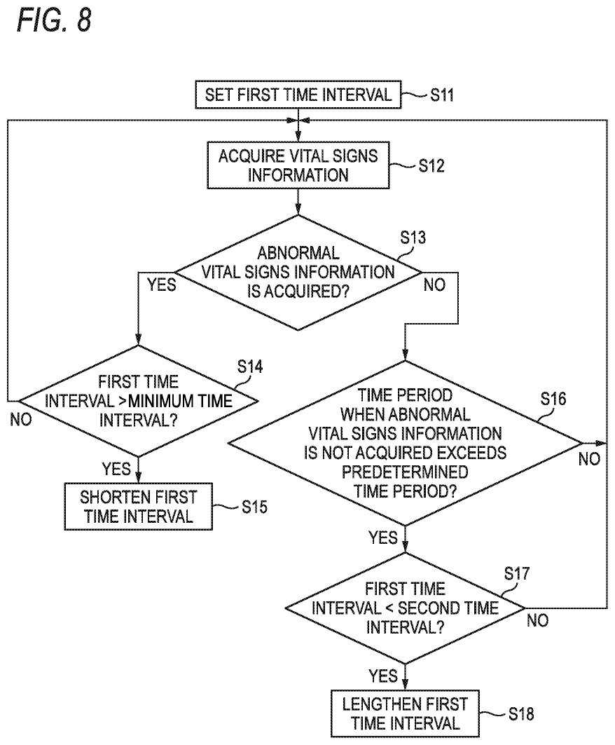

FIG. 8 illustrates a flowchart relating to setting of the first time interval. The embodiment will be described under assumption that the first time interval is 15 minutes, and the second time interval is 60 minutes. Steps S11 and S12 are same as or similar to Steps S1 and S2 illustrated in FIG. 7, and therefore their description is omitted. In step S13, the controller 2 determines whether the vital signs information acquired by the acquiring section 12 contains abnormal vital signs information indicating an abnormality of the patient, at a 15-minute interval (the first time interval). If YES in step S13, the controller 2 determines whether the first time interval exceeds the minimum time interval or not (step S14). In the embodiment, it is assumed that the minimum time interval is 5 minutes. If YES in step S14, the controller 2 sets the first time interval so as to be shortened (step S15). If NO in step S14, by contrast, the process returns to step S12. In the embodiment, the first time interval is 15 minutes, and therefore exceeds the minimum time interval (5-minute interval). Consequently, the controller 2 controls the first time interval so as to be shortened.

For example, the first time interval may be set depending on the color group. In the case of the white group, for example, the first time interval is 20 minutes. In the case of the green group, the first time interval is 15 minutes. In the case of the orange group, the first time interval is 10 minutes. In the case of the red group, the first time interval is 5 minutes. For example, it is assumed that, in the case where the apnea-hypopnea index is classified into the white group, and the first time interval is set to 20 minutes, the apnea rate and hypopnea rate that are measured at the first time interval (20 minutes) are 1 and 2, respectively. In this case, the estimated value of the apnea-hypopnea index per the second time interval (60 minutes) is 9. When the apnea-hypopnea index is 9, the color group is classified into the green group (see FIG. 6). Therefore, the first time interval is set to 15 minutes.

If NO in step S13, the controller 2 determines whether the time period when the abnormal vital signs information indicating an abnormality of the patient is not acquired exceeds a predetermined time period or not (step S16). If YES in step S16, the process advances to step S17. If No in step S16, by contrast, the process returns to step S12. In step S17, the controller 2 determines whether the first time interval is shorter than the second time interval or not. If YES in step S17, the controller 2 sets the first time interval to be lengthened (step S18). Among the first time intervals, the longest first time interval is identical with the second time interval. If YES in step S17, therefore, the first time interval is set to approach the second time interval. If NO in step S17, by contrast, the process returns to step S12. In the embodiment, the first time interval is a 15-minute interval, and therefore shorter than the second time interval (60-minute interval). In the embodiment, the time period when the abnormal vital signs information indicating an abnormality of the patient is not acquired exceeds the predetermined value, therefore, the controller 2 controls the first time interval so as to be lengthened.

For example, it is assumed that, in the case where the apnea-hypopnea index is classified into the green group, and the first time interval is set to 15 minutes, the apnea rate and hypopnea rate that are measured at the first time interval (15 minutes) are 0 and 1, respectively. In this case, the estimated value of the apnea-hypopnea index per the second time interval (60 minutes) is 4. When the apnea-hypopnea index is 4, the color group is classified into the white group (see FIG. 6). Therefore, the first time interval is set to 20 minutes.

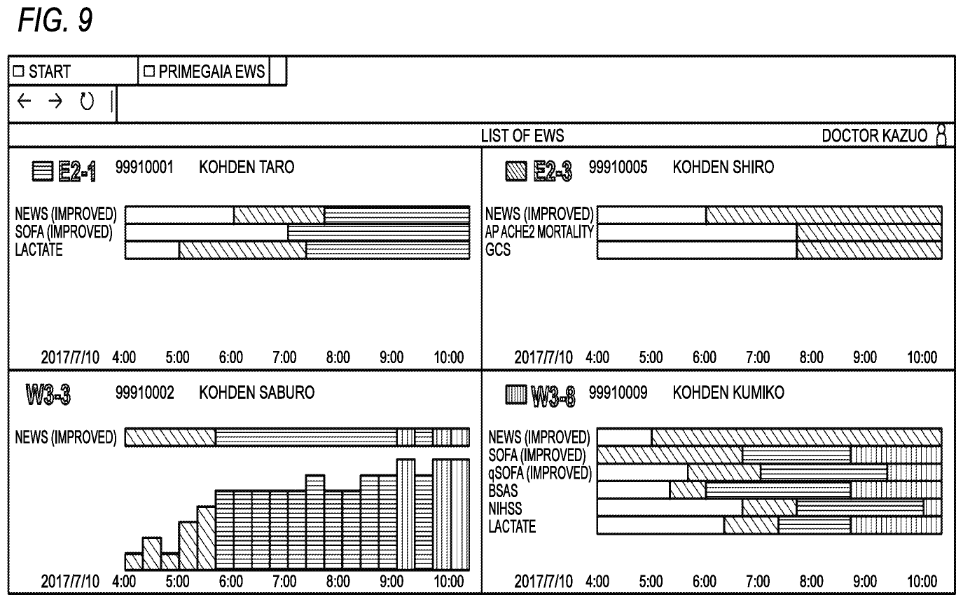

FIG. 9 is a view relating to a screen displaying a condition change of the patient. As illustrated in FIG. 9, a condition change of the patient is displayed in a display mode that is normalized, i.e., a colored display mode, on the displaying section 13 of the bedside monitor 10. In the embodiment illustrated in FIG. 9, hatching is applied in place of coloring. The hatching corresponding to the white group is plain. The hatching corresponding to the green group is represented by diagonals extending from the upper left to the lower right. The hatching corresponding to the orange group is represented by horizontal lines. The hatching corresponding to the red group is represented by vertical lines. The screen illustrated in FIG. 9 is partitioned into four zones. In the zones, sets of information of patients are displayed, respectively. In the screen example illustrated in FIG. 9, condition changes of four patients are illustrated in a graph-like manner.

In the middle portions of the zones, the names of the patients are displayed, respectively. An identification number of each of the patients is displayed on the left side of the patient name. A room number corresponding to the patient is displayed on the left side of the identification number. In the specification, information containing the patient name, the identification number, and the room number is sometimes referred to as patient identification information. The room number is colored by a color corresponding to the color group into which the condition change is classified based on the combined score or numerical values at the latest timing (10:00 of Jul. 10, 2017). The name(s) of determination method(s) and/or parameter(s) that are arbitrarily selected by the medical person are displayed below the room number. On the right side of each of the determination method(s) and/or the parameter(s), determination results corresponding to the determination method or the parameter are displayed. The determination results are displayed in time sequence. The determination results are displayed in a vertical bar graph or a horizontal bar graph. Each of the determination results is normalized i.e., colored based on the combined score or measurement value that corresponds to the determination method or the parameter. Therefore, the determination results are visually distinguished from one another.

The screen illustrated in FIG. 9 will be described in detail while paying attention to one of the patients, i.e., KOHDEN Taro. Information relating to KOHDEN Taro is displayed in the upper left zone. In the zone, information relating to the determination method (NEWS (improved)) based on NEWS, that relating to the determination method (SOFA (improved)) based on SOFA, and that relating to lactate are displayed in horizontal bar graphs. The horizontal bar graph relating to the determination method based on NEWS will be considered. When the combined score of the determination method based on NEWS of KOHDEN Taro from 4:00 of Jul. 10, 2017 to 5:40 of the same day is 0, the determining section 6 classifies the combined score of the determination method of KOHDEN Taro in the time band, into the white group. Therefore, the horizontal bar graph in the time band is colored by white. When the combined score of the determination method of KOHDEN Taro from 5:40 of the same day to 7:20 is 1 to 4, the determining section 6 classifies the combined score of the determination method of KOHDEN Taro in the time band, into the green group. Therefore, the horizontal bar graph in the time band is colored by green. When the combined score of the determination method of KOHDEN Taro after 7:20 of the same day is 5 and 6, the determining section 6 classifies the combined score of the determination method of KOHDEN Taro in the time band, into the orange group. Therefore, the horizontal bar graph in the time band is colored by orange.

The horizontal bar graph relating to the determination method based on SOFA will be considered. When the combined score of the determination method of KOHDEN Taro from 4:00 of Jul. 10, 2017 to 6:40 of the same day is 0, the determining section 6 classifies the combined score of the determination method of KOHDEN Taro in the time band, into the white group. When the combined score of the determination method of KOHDEN Taro after 6:40 of the same day is 1, the determining section 6 classifies the combined score of the determination method of KOHDEN Taro in the time band, into the orange group.

The horizontal bar graph relating to lactate will be considered. When the value of lactate of KOHDEN Taro from 4:00 of Jul. 10, 2017 to 4:40 of the same day is less than 1 mmol/L, the determining section 6 classifies the value of lactate of KOHDEN Taro in the time band into the white group. When the value of lactate of KOHDEN Taro from 4:40 of the same day to 7:00 is 1 mmol/l, the determining section 6 classifies the value of lactate of KOHDEN Taro in the time band into the green group. When the value of lactate of KOHDEN Taro after 7:00 of the same day is from 2 mmol/L to 4 mmol/L, the determining section 6 classifies the value of lactate of KOHDEN Taro in the time band into the orange group.

Then, the screen illustrated in FIG. 9 will be described in detail while paying attention to KOHDEN Saburo. Information relating to KOHDEN Saburo is displayed in the lower left zone. In the zone, information relating to the determination method (NEWS (improved)) based on NEWS is displayed in vertical and horizontal bar graphs. The horizontal bar graph is located above the vertical bar graph. As illustrated in the vertical bar graph, the combined score of the determination method of KOHDEN Saburo from 4:00 of Jul. 10, 2017 to 5:20 of the same day transits between 1 and 4. Consequently, the determining section 6 classifies the combined score of the determination method of KOHDEN Saburo in the time band, into the green group. Therefore, the vertical and horizontal bar graphs in the time band are colored by green. The combined score of the determination method of KOHDEN Saburo from 5:20 of the same day to 8:40 transits between 5 and 6. Consequently, the determining section 6 classifies the combined score of the determination method of KOHDEN Saburo in the time band, into the orange group. Therefore, the vertical and horizontal bar graphs in the time band are colored by orange. The combined score of the determination method of KOHDEN Saburo from 8:40 of the same day to 9:00 is 7. Consequently, the determining section 6 classifies the combined score of the determination method of KOHDEN Saburo in the time band, into the red group. Therefore, the vertical and horizontal bar graphs in the time band are colored by red. The combined score of the determination method of KOHDEN Saburo from 9:00 of the same day to 9:20 is 6. Consequently, the determining section 6 classifies the combined score of the determination method of KOHDEN Saburo in the time band, into the orange group. The combined score of the determination method of KOHDEN Saburo after 9:20 of the same day is 7. Consequently, the determining section 6 classifies the combined score of the determination method of KOHDEN Saburo in the time band, into the red group.

The display controller 8 controls the displaying section 13 of the bedside monitor 10 so as to display the room number colored by a color corresponding to the color group that, among the color groups relating to the combined score or numerical value at the latest timing (10:00 of Jul. 10, 2017), indicates the worst patient condition level. The control will be described by exemplifying KOHDEN Kumiko. The color group to which the combined score or measurement values of KOHDEN Kumiko belongs at the latest timing are the green and red groups. The patient condition level in the red group is worse than that in the green group, and therefore the room number of KOHDEN Kumiko is displayed on the displaying section 13 in a state where the room number is colored by the color corresponding to the red group, i.e., red.

Although, in the zone of KOHDEN Taro, information relating to the determination methods and the parameter (lactate) is displayed, only the determination methods may be displayed as in the display mode in the zone of KOHDEN Shiro.

Although, in the screen illustrated in FIG. 9, the sets of information respectively relating to the four patients are displayed on the one screen, the display manner is not limited to this example. For example, only a set of information relating to one patient may be displayed on the one screen.

Although, in the screen illustrated in FIG. 9, the screen is divided in two horizontal rows and two vertical columns, the division manner is not limited to this example. For example, the screen may be divided in one horizontal row and four vertical columns. Alternatively, the displaying section 13 may be configured so that the display mode is appropriately changed by the display controller 8 in accordance with the size of the displaying portion or the like.

Some methods for determining the condition of a patient include the respiration rate as an evaluation item. As a method for measuring the respiration rate, known are a method in which a medical person visually measures the respiration rate, the impedance method using an electrocardiogram, and the measurement method using the carbon dioxide concentration (EtCO2). In the case where a medical person visually measures the respiration rate of a patient, the medical person must stay near the patient during the measurement of the respiration rate. Therefore, there is a possibility that the operational efficiency of the medical person is lowered. In the case where the impedance method or the measurement method using the carbon dioxide concentration (EtCO2) is employed, by contrast, these methods are susceptible to noise such as artifact, and therefore it is difficult to accurately measure the respiration rate.

The apnea-hypopnea index is known as an index representing the condition (quality) of the respiration. As described above, the apnea-hypopnea index is a value that is calculated by converting the total number of the apnea rate and hypopnea rate during sleep, to the mean rate per hour. The apnea-hypopnea index is a parameter that is used mainly in determination of the severity of sleep apnea syndrome. When the apnea-hypopnea index is added to parameters of a determination method in which the respiration rate is used as a parameter in determination of a condition change of the patient, therefore, a condition change of the patient can be determined also in consideration of the quality of the respiration of the patient, and hence a condition change of the patient can be accurately determined.

According to the program, condition change determination method, and condition change determination apparatus 1 that are configured as described above, a condition change of the patient is determined based on at least one of the apnea rate and the hypopnea rate. Therefore, a condition change of the patient can be determined in consideration of the quality of the respiration. As a result, the condition change of the patient can be accurately determined.

According to the program, condition change determination method, and condition change determination apparatus 1 that are configured as described above, a condition change of the patient can be determined based not only on the apnea rate and the hypopnea rate, but also on the respiration rate. Therefore, the condition change of the patient can be more accurately determined.

According to the program, condition change determination method, and condition change determination apparatus 1 that are configured as described above, at least one of the apnea rate and the hypopnea rate is measured at the first time interval. When at least one of the apnea rate and the hypopnea rate is measured, an estimated value of at least one of the apnea and the hypopnea rates per the second time interval which is longer than the first time interval is calculated. When the time intervals are shortened, for example, a condition change of the patient can be therefore rapidly determined.

According to the program, condition change determination method, and condition change determination apparatus 1 that are configured as described above, the first time interval is set based on at least one of the apnea rates, hypopnea rates and vital signs information other than the apnea and hypopnea rates. In the case where abnormal vital signs information is acquired, for example, the first time interval is automatically set to be shorter, and therefore a condition change of the patient can be rapidly determined. As a result, the medical person can rapidly know the condition change of the patient, and promptly take countermeasures.

According to the program, condition change determination method, and condition change determination apparatus 1 that are configured as described above, when vital signs information indicating an abnormality of the patient is acquired, the first time interval is set be shorter than the time interval that is set before the acquisition of the vital signs information indicating the abnormality. When abnormal vital signs information is acquired, therefore, the first time interval is automatically set to be shorter, and the condition of the patient can be rapidly determined. As a result, the medical person can rapidly know the condition change of the patient, and promptly take countermeasures.

According to the program, condition change determination method, and condition change determination apparatus 1 that are configured as described above, in the case where vital signs information indicating an abnormality of the patient is not acquired within a predetermined time period, the first time interval is set to approach the second time interval that is longer than the first time interval. As a result, a condition change of the patient is determined at an adequate time interval.

According to the program, condition change determination method, and condition change determination apparatus 1 that are configured as described above, a condition change of the patient is determined based on the respiration rate and apnea and hypopnea rates that are acquired in a measurement by the impedance method using an electrocardiogram, or a measurement of the carbon dioxide concentration (EtCO2). Therefore, a condition change of the patient can be accurately determined while shortening the time period for measuring the respiration rate.

According to the program, condition change determination method, and condition change determination apparatus 1 that are configured as described above, a result of the determination relating to a condition change of the patient is displayed in a display mode that is normalized under predetermined normalization conditions. When the medical person checks the determination result that is displayed on the displaying section in the normalized display mode, therefore, the medical person can evaluate determination results by using a constant criterion.

According to the configuration, as described above, the medical person can check a determination results by using a constant criterion, and therefore the burden imposed on the medical person can be reduced.

According to the program, condition change determination method, and condition change determination apparatus 1 that are configured as described above, a plurality of determination results relating to a condition change of the patient are displayed in a display mode that is normalized under predetermined normalization conditions. Therefore, the medical person can check the determination results that are displayed on the displaying section in the normalized display mode, and hence evaluate the determination results in a uniform manner.

According to the configuration, in the case where the medical person wishes to comprehensively determine a condition change of the patient by using a plurality of determination methods, therefore, the burden imposed on the medical person can be reduced.

### Second Embodiment

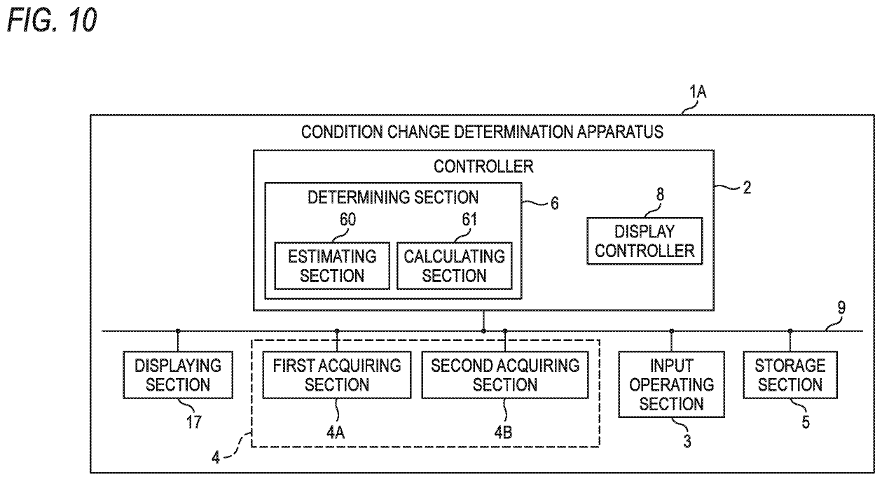

As illustrated in FIG. 10, a condition change determination apparatus 1A of a second embodiment is different from the condition change determination apparatus 1 of the first embodiment in that the apparatus 1A includes an input operating section 3, an acquiring section 4, and a displaying section 17. In the same or similar manner as the acquiring section 12, the acquiring section 4 may include a first acquiring section 4A and a second acquiring section 4B. In the second embodiment, when the condition change determination apparatus 1A executes processes that are same as or similar to those executed by the condition change determination apparatus 1 and bedside monitor 10 in the first embodiment, a screen illustrated in FIG. 9 is displayed on the displaying section 17.

Although, in the above, the embodiment in which the scores and the combined score are expressed by numerals has been described, the invention is not limited to the example. For example, the scores and the combined score may be expressed by degrees that cannot be expressed by using numerals, such as the A evaluation.

Although, in the above, the embodiment in which coloring is employed as the method of visually distinguishing determination results from one another has been described, the invention is not limited to the example. For example, a method in which results are visually distinguished from one another by arbitrary hatching may be employed.

Although the first embodiment in which the display controller 8 of the controller 2 controls the displaying section 13 has been described, the invention is not limited to the example. For example, the monitor controller 15 may produce image data that are to be displayed on the displaying section 13, based on information acquired from the controller 2. Alternatively, the monitor controller 15 may control the displaying section 13 so as to display image data produced by the display controller 8. Furthermore, the monitor controller 15 may control the displaying section 13 so as to display waveform data and the like, based on the vital signs information acquired by the acquiring section 12.

The condition change determination apparatuses 1, 1A of the embodiments may be communicable with another vital signs information measurement apparatus, a computer for a medical person, or the like.

Although, in the above, the embodiment in which step S3, and step S4 or S5 are executed has been described, the invention is not limited to the example. For example, step S3 and step S4 may not be executed, and only step S5 may be executed. When step S2 is executed, namely, step S5 may be then executed. In this case, even when the apnea and hypopnea rates are measured at an interval that is equal to or longer than the second time interval, an estimated value of the apnea-hypopnea index is calculated by the estimating section 60. As a result, even when the apnea and hypopnea rates are measured at an interval that is equal to or longer than the second time interval, the condition of the patient is determined based on the estimated value of the apnea-hypopnea index.

The presently disclosed subject matter is not limited to the above-described embodiments, and may be freely subjected to modifications, improvements, and the like. In addition, the materials, shapes, dimensions, values, forms, numbers, installation places, and the like of the components of the above-described embodiments are arbitrary and not limited insofar as the presently disclosed subject matter can be achieved.

According to the program having the above-described configuration, a condition change of the patient is determined based on at least one of the apnea and hypopnea rates. Therefore, a condition change of the patient can be determined in consideration of the quality of the respiration.

According to the configuration, consequently, it is possible to provide a program that can accurately determine a condition change of a patient in consideration of the quality of the respiration.

A condition change determination method of a mode of the presently disclosed subject matter includes the steps:

acquiring at least one of apnea and hypopnea rates of a patient;

acquiring vital signs information of the patient other than the apnea and hypopnea rates; and

determining a condition change of the patient by a determination method for determining a condition change of a patient based on at least one of the apnea and hypopnea rates, and the vital signs information other than the apnea and hypopnea rates.

A condition change determination apparatus of a mode of the presently disclosed subject matter includes a determining section that determines a condition change of a patient by a determination method for determining a condition change of a patient based on vital signs information that includes: at least one of apnea and hypopnea rates of the patient; and vital signs information of the patient other than the apnea and hypopnea rates.

The determining section determines a condition change of the patient based on the at least one of the apnea and hypopnea rates, and the vital signs information other than the apnea and hypopnea rates.

According to the presently disclosed subject matter, it is possible to provide a condition change determination method and condition change determination apparatus that, when a medical person uses a method for determining a condition change of a patient, such as NEWS, can accurately determine the condition of the patient, and also to provide a program and computer readable medium that are to be used in the apparatus or the method.

What is claimed is:

1. A non-transitory computer readable medium including a program that causes a computer to realize the functions of:
acquiring at least one of apnea and hypopnea rates of a patient;
acquiring vital signs information of the patient other than the apnea and hypopnea rates; and
determining a condition change of the patient by a determination method for determining a condition change of a patient based on at least one of the apnea and hypopnea rates, and the vital signs information other than the apnea and hypopnea rates.

2. The non-transitory computer readable medium according to claim 1,
wherein at least a score indicating a condition of the patient is calculated based on at least one of the apnea and hypopnea rates, and
wherein a condition change of the patient is determined based on the calculated score.

3. The non-transitory computer readable medium according to claim 1,
wherein the vital signs information other than the apnea and hypopnea rates includes a respiration rate.

4. The non-transitory computer readable medium according to claim 1 further comprises a function of, based on at least one of apnea and hypopnea rates that are measured at a first time interval, calculating an estimated value of at least one of apnea and hypopnea rates per a second time interval that is longer than the first time interval.

5. The non-transitory computer readable medium according to claim 4,
wherein the first time interval is set based on at least one of the apnea rates, hypopnea rates and the vital signs information other than the apnea and hypopnea rates.

6. The non-transitory computer readable medium according to claim 5,
wherein when abnormal vital signs information indicating an abnormality of the patient is acquired, the first time interval is set to be shorter than a time interval that is set before acquisition of the abnormal vital signs information.

7. The non-transitory computer readable medium according to claim 5,
wherein when abnormal vital signs information indicating an abnormality of the patient is not acquired within a predetermined time period, the first time interval is set to approach the second time interval.

8. The non-transitory computer readable medium according to claim 3,
wherein the respiration rate is acquired by a measurement by an impedance method using an electrocardiogram, or a measurement of a carbon dioxide concentration (EtCO2).

9. The non-transitory computer readable medium according to claim 1,
wherein the program further includes a function of displaying a determination result relating to the determined condition change of the patient, in a display mode that is normalized under predetermined normalization conditions.

10. The non-transitory computer readable medium according to claim 9,
wherein the function of determining a condition change of the patient has a plurality of determination methods, and
wherein determination results that are determined by the plurality of determination methods are normalized under predetermined normalization conditions.

11. A condition change determination method comprising:
acquiring at least one of apnea and hypopnea rates of a patient;
acquiring vital signs information of the patient other than the apnea and hypopnea rates; and
determining a condition change of the patient by a determination method for determining a condition change of a patient based on at least one of the apnea and hypopnea rates, and the vital signs information other than the apnea and hypopnea rates.

12. The condition change determination method according to claim 11, wherein the vital signs information other than the apnea and hypopnea rates includes a respiration rate.

13. A condition change determination apparatus comprising:
a determining section that determines a condition change of a patient by a determination method for determining a condition change of a patient based on vital signs information that includes at least one of apnea and hypopnea rates of the patient and vital signs information of the patient other than the apnea and hypopnea rates,
wherein the determining section determines a condition change of the patient based on the at least one of the apnea and hypopnea rates, and the vital signs information other than the apnea and hypopnea rates.

14. The condition change determination apparatus according to claim 13, wherein the vital signs information other than the apnea and hypopnea rates includes a respiration rate.
